(19) United States
(12) Patent Application Publication
(10) Pub. No.: US 2024/0085391 A1
(43) Pub. Date: Mar. 14, 2024

(54) GAS DETECTION METHOD, PROGRAM, CONTROL SYSTEM, AND GAS DETECTION SYSTEM

(71) Applicant: Panasonic Intellectual Property Management Co., Ltd., Osaka (JP)

(72) Inventor: Hiroshi USHIO, Osaka (JP)

(21) Appl. No.: 18/261,172

(22) PCT Filed: Jan. 17, 2022
(86) PCT No.: PCT/JP2022/001419

§ 371 Date: Jul. 12, 2023

(30) Foreign Application Priority Data
Jan. 21, 2021 (JP) 2021-008310

(51) Int. Cl. G01N 33/00

(52) U.S. Cl. CPC G01N 33/0014; G01N 33/0073; G01N 2035/00475

(57) ABSTRACT

A gas detection method includes a first process, a second process, and a third process. The first to third process include changing routes to be followed by gases into first to third routes, respectively. The first route follows a first passage, a sensor chamber, and a second passage in this order. The second route follows a sample gas supply path, which bypasses a filter unit, and the sensor chamber in this order and allows the gas to be exhausted without letting the gas pass through the filter unit while flowing from the sensor chamber. The third route follows the first passage and the sensor chamber in this order and allows the gas to be exhausted without letting the gas pass through the filter unit while flowing from the sensor chamber.

### TECHNICAL FIELD

The present disclosure generally relates to a gas detection method, a program, a control system, and a gas detection system. More particularly, the present disclosure relates to a gas detection method, a program, a control system, and a gas detection system, all of which are configured or designed to detect molecules as a detection target in a sample gas.

### BACKGROUND ART

Patent Literature 1 discloses a gas detection device (gas detection system) that alternately introduces a referential gas (reference gas) and a gas under test (sample gas) into a sensor (gas sensor) in a housing to detect a particular component (molecules as a detection target) in the gas under test. According to Patent Literature 1, the gas under test is passed through, and purified by, a purification means (filter unit), thereby generating the referential gas. The referential gas thus generated is supplied to the sensor.

### CITATION LIST

### Patent Literature

Patent Literature 1: JP 2004-53582 A

### SUMMARY OF INVENTION

An object of the present disclosure is to provide a gas detection method, a program, a control system, and a gas detection system, all of which are configured or designed to reduce deterioration of a filter unit.

A gas detection method according to an aspect of the present disclosure is designed to use a gas detection mechanism. The gas detection mechanism includes a filter unit and a sensor chamber. The filter unit has a first passage and a second passage as a gas flow channel. The filter unit selectively transfers a component to be removed in a gas from the first passage to the second passage. The sensor chamber houses a gas sensor. The gas detection method includes a first process, a second process, and a third process. The first process includes changing routes to be followed, from inlet to outlet, by a reference gas into a first route. The reference gas provides a reference concentration for detection target molecules in a sample gas. The first route follows the first passage, the sensor chamber, and the second passage in this order. The second process includes changing routes to be followed, from inlet to outlet, by the sample gas into a second route. The second route follows a sample gas supply path, which bypasses the filter unit, and the sensor chamber in this order and allows the sample gas to be exhausted without letting the sample gas pass through the filter unit while flowing from the sensor chamber. The third process is performed after the second process and before the first process. The third process includes changing routes to be followed, from inlet to outlet, by the reference gas into a third route. The third route follows the first passage and the sensor chamber in this order and allows the reference gas to be exhausted without letting the reference gas pass through the filter unit while flowing from the sensor chamber.

A program according to another aspect of the present disclosure is designed to cause one or more processors to perform the gas detection method described above.

A control system according to still another aspect of the present disclosure controls a gas detection mechanism. The gas detection mechanism includes a filter unit and a sensor chamber. The filter unit has a first passage and a second passage as a gas flow channel. The filter unit selectively transfers a component to be removed in a gas from the first passage to the second passage. The sensor chamber houses a gas sensor. The control system includes a control unit that performs a first process, a second process, and a third process. The first process includes changing routes to be followed, from inlet to outlet, by a reference gas into a first route. The reference gas provides a reference concentration for detection target molecules in a sample gas. The first route follows the first passage, the sensor chamber, and the second passage in this order. The second process includes changing routes to be followed, from inlet to outlet, by the sample gas into a second route. The second route follows a sample gas supply path, which bypasses the filter unit, and the sensor chamber in this order and allows the sample gas to be exhausted without letting the sample gas pass through the filter unit while flowing from the sensor chamber. The third process is performed after the second process and before the first process. The third process includes changing routes to be followed, from inlet to outlet, by the reference gas into a third route. The third route follows the first passage and the sensor chamber in this order and allows the reference gas to be exhausted without letting the reference gas pass through the filter unit while flowing from the sensor chamber.

A gas detection system according to yet another aspect of the present disclosure includes the control system described above and the gas detection mechanism described above.

### BRIEF DESCRIPTION OF DRAWINGS

FIG. 1 illustrates a schematic system configuration for a gas detection system according to an exemplary embodiment;

FIG. 2 is a timing chart showing how the gas detection system performs a gas detection operation;

FIG. 3 is a graph showing how humidity changes in the gas detection system;

FIG. 4 is a graph showing a relationship between the maximum humidity in the gas detection system and the length of an exhaust period;

FIG. 5 illustrates a schematic system configuration for a gas detection system according to a first variation; and

FIG. 6 illustrates a schematic system configuration for a gas detection system according to a second variation.

### DESCRIPTION OF EMBODIMENTS

A gas detection method, a program, a control system, and a gas detection system according to an exemplary embodiment will be described with reference to the accompanying drawings. Note that the embodiment to be described below is only an exemplary one of various embodiments of the present disclosure and should not be construed as limiting. Rather, the exemplary embodiment may be readily modified in various manners depending on a design choice or any other factor without departing from the scope of the present disclosure. The drawings to be referred to in the following description of embodiments are all schematic representations. Thus, the ratio of the dimensions (including thicknesses) of respective constituent elements illustrated on the drawings does not always reflect their actual dimensional ratio.

### Embodiment

### (1) Overview

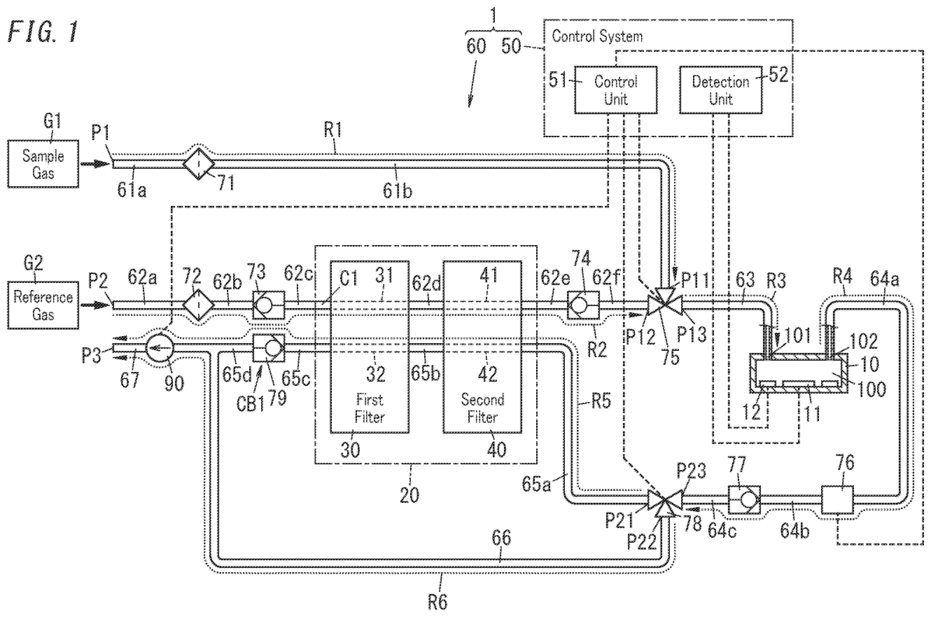

FIG. 1 illustrates a schematic system configuration for a gas detection system 1 according to an exemplary embodiment.

The gas detection system 1 according to this embodiment may be used to, for example, detect odor molecules as detection target molecules. Examples of the odor molecules include volatile organic compounds (VOCs) and ammonia. The gas detection system 1 according to this embodiment is used to detect VOCs as detection target molecules. The gas detection system 1 according to this embodiment detects VOCs as odor molecules included in a sample gas G1 such as a gas taken from a food, a breath taken from a human body, or the air taken from a building room. Note that the detection target molecules to be detected by the gas detection system 1 do not have to be VOCs but may also be multiple types of odor molecules including VOCs or non-odor molecules such as molecules of a flammable gas or a poisonous gas like carbon monoxide.

The gas detection system 1 according to this embodiment includes a control system 50 and a gas detection mechanism 60.

The control system 50 controls the gas detection mechanism 60. The gas detection mechanism 60 includes a filter unit 20 and a sensor chamber 100. The filter unit 20 has first passages 31, 41 and second passages 32, 42 as a gas flow channel. The filter unit 20 selectively transfers a component to be removed in a gas from the first passage 31 to the second passage 32 (and from the first passage 41 to the second passage 42). The sensor chamber 100 houses a gas sensor 11.

The control system 50 includes a control unit 51 that performs a first process, a second process, and a third process. The first process includes changing routes to be followed, from inlet to outlet, by a reference gas G2 into a first route. The reference gas G2 provides a reference concentration for detection target molecules in a sample gas G1. The first route includes a reference gas supply path R2, a first relay path R3, a second relay path R4, and a first exhaust path R5. The first route follows the first passages 31 (and 41), the sensor chamber 100, and the second passages 32 (and 42) in this order.

The second process includes changing routes to be followed, from inlet to outlet, by the sample gas G1 into a second route. The second route includes a sample gas supply path R1, the first relay path R3, the second relay path R4, and a second exhaust path R6. The second route follows the sample gas supply path R1, which bypasses the filter unit 20, and the sensor chamber 100 in this order and allows the sample gas to be exhausted without letting the sample gas pass through the filter unit 20 while flowing from the sensor chamber 100.

The third process is performed after the second process and before the first process. The third process includes changing routes to be followed, from inlet to outlet, by the reference gas G2 into a third route. The third route includes the reference gas supply path R2, the first relay path R3, the second relay path R4, and the second exhaust path R6. The third route follows the first passages 31 (and 41) and the sensor chamber 100 in this order and allows the reference gas to be exhausted without letting the reference gas pass through the filter unit 20 while flowing from the sensor chamber 100.

If the first process were performed after the second process with the third process omitted, then the sample gas G1 remaining in the sensor chamber 100 would be exhausted via the second passages 32 (and 42) of the filter unit 20. Unlike the reference gas G2, the sample gas G1 remaining in the sensor chamber 100 has not passed through the first passages 31 (and 41), and therefore, includes the component to be removed at a relatively high concentration. Thus, letting the sample gas G1 pass through the second passage 32 (and 42) would cause the filter unit 20 to deteriorate.

In contrast, according to this embodiment, performing the third process after the second process allows the sample gas G1, remaining in the sensor chamber 100, to be replaced with the reference gas G2. This reduces the chances of the sample gas G1, remaining in the sensor chamber 100, passing through the second passages 32 (and 42) of the filter unit 20, thus reducing deterioration of the filter unit 20. This stabilizes the quality of the reference gas G2 that provides a reference concentration for the detection target molecules, thus allowing the concentration of the detection target molecules in the sample gas G1 to be measured more accurately.

In addition, performing the third process allows the sample gas G1, remaining in the sensor chamber 100, to be replaced with the reference gas G2. This reduces the chances of the component to be removed in the sample gas G1, remaining in the sensor chamber 100, being detected by the gas sensor 11 while the first process is being performed. Consequently, the concentration of the detection target molecules in the sample gas G1 may be measured more accurately.

A gas detection method according to this embodiment includes the first process, the second process, and the third process as described above.

A program according to this embodiment is designed to cause one or more processors to perform the gas detection method described above.

### (2) Details

A gas detection system 1 according to an exemplary embodiment will be described in detail with reference to the accompanying drawings.

(2.1) Configuration

The gas detection mechanism 60 of the gas detection system 1 according to this embodiment includes: a gas sensor 11 for detecting detection target molecules in the sample gas G1; a water content measuring unit 12 for measuring the content of water in the gas in the sensor chamber 100; the reference gas supply path R2; and the sample gas supply path R1. The reference gas supply path R2 is a flow channel that allows the reference gas G2, providing a reference concentration for the detection target molecules, to pass through the filter unit 20 for reducing the target component to be removed in the reference gas G2 before being supplied to the sensor chamber 100 that houses the gas sensor 11. The sample gas supply path R1 is a flow channel that allows the sample gas G1 to be supplied to the sensor chamber 100 without letting the sample gas G1 pass through the filter unit 20.

As used herein, the “flow channel” in the gas detection mechanism 60 refers to a path, through which a gas such as the sample gas G1 and the reference gas G2 flows, and which is made up of piping, valves, and other members.

In the gas detection system 1 according to this embodiment, the sample gas G1 is supplied to the sensor chamber 100 through the sample gas supply path R1 that bypasses the filter unit 20. This reduces the deterioration of the filter unit 20 that would be caused by letting the sample gas G1 pass through the filter unit 20. This stabilizes the quality of the reference gas G2 that provides a reference concentration for the detection target molecules, thus reducing a dispersion in the results of detection of the sample gas G1.

In addition, the gas detection mechanism 60 further includes: a first air inlet port P1 (hereinafter sometimes referred to as a “sample gas inlet port”), through which the sample gas G1 is introduced from outside of the gas detection mechanism 60; a second air inlet port P2 (hereinafter sometimes referred to as a “reference gas inlet port”), through which the reference gas G2 is introduced from outside of the gas detection mechanism 60; and a gas outlet port P3, through which the gas inside the gas detection mechanism 60 is exhausted to the outside. In this embodiment, the air in the environment surrounding the gas detection mechanism 60 is used as the reference gas G2. As the sample gas G1, either the air in the environment surrounding the gas detection mechanism 60 (such as the air taken from a room in a building) or a gas taken from a food may be used, whichever is appropriate.

The gas detection mechanism 60 further includes an air pump 90. Making the air pump 90 suck in a gas through either the first air inlet port P1 or the second air inlet port P2 causes the gas sucked in through the first air inlet port P1 or the second air inlet port P2 to be supplied to the gas sensor 11 and then exhausted through the gas outlet port P3 to the outside.

Meanwhile, the control system 50 of the gas detection system 1 has at least the functions of the control unit 51 and a detection unit 52. The control unit 51 controls the operations of a plurality of solenoid valves (including a first branch valve 75, a second branch valve 78, and a proportional control solenoid valve 76) included in the gas detection mechanism 60 and the operation of the air pump 90. The detection unit 52 detects, based on the output value of the gas sensor 11, detection target molecules (e.g., VOCs in this embodiment) in the gas. The detection unit 52 also detects, based on the output value of the water content measuring unit 12, the content of water in the gas.

The gas detection mechanism 60 further includes a housing 10 including the sensor chamber 100 that houses the gas sensor 11 and the water content measuring unit 12.

The gas sensor 11 includes a sensing unit such as an electrochemical sensing unit, a semiconductor sensing unit, a field effect transistor sensing unit, a surface acoustic wave sensing unit, a crystal oscillator sensing unit, or a variable resistance sensing unit. The sensing unit has sensitivity to the detection target molecules (e.g., VOCs in this embodiment) and may have, for example, a resistance value that varies according to the concentration of the detection target molecules. The detection unit 52 extracts, as either a voltage signal or a current signal, the resistance value of the sensing unit included in the gas sensor 11 and detects, based on the resistance value of the sensing unit, the detection target molecules in the gas in the sensor chamber 100.

The water content measuring unit 12 measures the content of water in the gas in the sensor chamber 100. The content of water in the gas may be determined by measuring the temperature and humidity of the gas. The water content measuring unit 12 according to this embodiment includes a temperature-humidity sensor, which measures the temperature and humidity of the gas in the sensor chamber 100. Then, the water content measuring unit 12 measures, based on the temperature and humidity thus measured, the content of water in the gas, and outputs the content of water thus measured to the detection unit 52. In other words, the process of measuring the content of water in the reference gas G2 includes measuring the content of water in the reference gas G2 based on the results of measurement of the temperature and humidity of the reference gas G2 that has passed through the filter unit 20.

The housing 10 may be formed in the shape of a box out of a material such as a synthetic resin or a metal. The housing 10 has a first port 101, through which a gas (which is either the sample gas G1 or the reference gas G2) is introduced from outside of the housing 10 into the sensor chamber 100, and a second port 102, through which the gas is exhausted from the sensor chamber 100 to the outside of the housing 10.

The first air inlet port P1 is connected to an input port of a particle filter 71 via a pipe 61a. An output port of the particle filter 71 is connected to a first input port P11 of the first branch valve 75 (three-way solenoid valve) via a pipe 61b. To introduce the sample gas G1, either a syringe or collecting bag, each containing the sample gas G1, may be connected to the first air inlet port P1 or a source of generation of the sample gas G1 may be brought closer to the first air inlet port P1. The sample gas G1 introduced into the first air inlet port P1 will have dust and other particles with a relatively large particle size filtered out by the particle filter 71 and then be introduced into the first input port P11 of the first branch valve 75.

The second air inlet port P2 is connected to an input port of a particle filter 72 via a pipe 62a. An output port of the particle filter 72 is connected to an input port of a check valve 73 via a pipe 62b. An output port of the check valve 73 is connected to the filter unit 20 via a pipe 62c.

The filter unit 20 is provided to reduce the target component to be removed in the reference gas G2 that has been introduced through the second air inlet port P2.

In this embodiment, the filter unit 20 includes multiple types of target-by-target filters, which are provided to reduce mutually different components to be removed. The filter unit 20, including such multiple types of target-by-target filters, may reduce multiple types of components to be removed by using the multiple types of target-by-target filters. In this embodiment, the filter unit 20 may remove, for example, VOCs which are detection target molecules for the gas sensor 11, and water that affects the measurement results of the gas sensor 11. For this purpose, the filter unit 20 includes, as the multiple types of target-by-target filters, a first filter 30 for reducing the detection target molecules in the gas (e.g., VOCs in this embodiment) and a second filter 40 for reducing water in the gas. The first filter 30 and the second filter 40 are provided to reduce mutually different components to be removed. Thus, providing these multiple types of target-by-target filters for the filter unit 20 allows the filter unit 20 to reduce multiple types of components to be removed (e.g., VOCs and water in this embodiment). Note that the multiple types of target-by-target filters provided for the filter unit 20 do not have to consist of the first filter 30 for reducing VOCs and the second filter 40 for reducing water. Optionally, the filter unit 20 may include at least one target-by-target filter for reducing a component to be removed other than VOCs and water. Examples of such components to be removed include ammonia, hydrogen sulfide, oxygen, carbon dioxide, and nitrogen.

Each of the first filter 30 and the second filter 40 includes a separation membrane including a hollow fiber. That is to say, the filter unit 20 includes separation membranes.

The first filter 30 includes a first passage 31, through which a gas flowing from the second air inlet port P2 toward the sensor chamber 100 passes, and a second passage 32 (i.e., a so-called “purge line”), through which a gas flowing from the sensor chamber 100 toward the gas outlet port P3 passes. Inside the first filter 30, the separation membrane is disposed between the first passage 31 and the second passage 32. In other words, the separation membrane separates the first passage 31 and the second passage 32 from each other. The first filter 30 for reducing the VOCs may include, for example, a separation membrane formed out of a hollow fiber of a silicone-based synthetic resin, for example.

Likewise, the second filter 40 also includes a first passage 41, through which the gas flowing from the second air inlet port P2 toward the sensor chamber 100 passes, and a second passage 42 (i.e., a so-called “purge line”), through which the gas flowing from the sensor chamber 100 toward the gas outlet port P3 passes. Inside the second filter 40, the separation membrane is disposed between the first passage 41 and the second passage 42. In other words, the separation membrane separates the first passage 41 and the second passage 42 from each other. The second filter 40 for reducing water may include, for example, a separation membrane formed out of a hollow fiber of a fluorine-based synthetic resin, for example.

One end of the first passage 31 of the first filter 30 is connected to the output port of the check valve 73 via a pipe 62c. The other end of the first passage 31 is connected to one end of the first passage 41 of the second filter 40 via a pipe 62d. The other end of the first passage 41 of the second filter 40 is connected to an input port of a check valve 74 via a pipe 62e. An output port of the check valve 74 is connected to a second input port P12 of the first branch valve 75 via a pipe 62f. An output port P13 of the first branch valve 75 is connected to the first port 101 of the housing 10 via a pipe 63.

The second port 102 of the housing 10 is connected to an input port of the proportional control solenoid valve 76 via a pipe 64a. An output port of the proportional control solenoid valve 76 is connected to an input port of a check valve 77 via a pipe 64b. The proportional control solenoid valve 76 is a variable orifice, of which the valve opening is adjustable. The valve opening of the proportional control solenoid valve 76 is controlled in accordance with an electrical signal (such as a current signal) supplied from the control unit 51. An output port of the check valve 77 is connected to an input port P23 of the second branch valve 78 via a pipe 64c. A first output port P21 of the second branch valve 78 is connected to one end of the second passage 42 of the second filter 40 via a pipe 65a. The other end of the second passage 42 of the second filter 40 is connected to one end of the second passage 32 of the first filter 30 via a pipe 65b. The other end of the second passage 32 is connected to an input port of a check valve 79 via a pipe 65c. An output port of the check valve 79 is connected to an air inlet port of the air pump 90 via a pipe 65d. An air outlet port of the air pump 90 is connected to the gas outlet port P3 via a pipe 67.

Also, a second output port P22 of the second branch valve 78 is coupled to the pipe 65d via a pipe 66.

In this embodiment, each of the pipes 61a, 61b, 62a-62f, 63, 64a-64c, 65a-65d, 66, and 67 may be either a pipe made of a synthetic resin or a metal or a flexible tube made of a synthetic resin, whichever is appropriate. The gas detection system 1 according to this embodiment is formed by housing the pneumatic circuit (i.e., the gas detection mechanism 60) and the control system 50 shown in FIG. 1 in a housing.

The gas detection mechanism 60 according to this embodiment includes, as paths for supplying the gases to the sensor chamber 100: the sample gas supply path R1 for supplying the sample gas G1 from the first air inlet port P1 to the first branch valve 75; the reference gas supply path R2 for supplying the reference gas G2 from the second air inlet port P2 to the first branch valve 75; and the first relay path R3 for supplying the sample gas G1 and the reference gas G2 from the first branch valve 75 to the sensor chamber 100.

The sample gas supply path R1 is a flow channel that leads from the first air inlet port P1 to the first branch valve 75 by following the pipe 61a, the particle filter 71, and the pipe 61b in this order.

The reference gas supply path R2 is a flow channel that leads from the second air inlet port P2 to the first branch valve 75 by following the pipe 62a, the particle filter 72, the pipe 62b, the check valve 73, the pipe 62c, the first passage 31 of the first filter 30, the pipe 62d, the first passage 41 of the second filter 40, the pipe 62e, the check valve 74, and the pipe 62f in this order.

The first relay path R3 is a flow channel leading from the first branch valve 75 to the sensor chamber 100 via the pipe 63.

The gas detection mechanism 60 includes, as paths for exhausting the gases from the sensor chamber 100: the second relay path R4 for supplying the sample gas G1 and the reference gas G2 from the sensor chamber 100 to the second branch valve 78; the first exhaust path R5 for exhausting the gases from the second branch valve 78 to the outside via the filter unit 20 and the gas outlet port P3; and the second exhaust path R6 for exhausting the gases from the second branch valve 78 to the outside through the gas outlet port P3 while bypassing the filter unit 20.

The second relay path R4 is a route that leads from the sensor chamber 100 to the second branch valve 78 by following the pipe 64a, the proportional control solenoid valve 76, the pipe 64b, the check valve 77, and the pipe 64c in this order.

The first exhaust path R5 is a flow channel for exhausting the gases from the second branch valve 78 to the outside through the gas outlet port P3 by letting the gases pass through the pipe 65a, the second passage 42 of the second filter 40, the pipe 65b, the second passage 32 of the first filter 30, the pipe 65c, the check valve 79, the pipe 65d, the air pump 90, and pipe 67 in this order.

The second exhaust path R6 is a flow channel for exhausting the gases from the second branch valve 78 to the outside through the gas outlet port P3 by letting the gases pass through the pipe 66, the pipe 65d, the air pump 90, and the pipe 67 in this order.

The first route formed by the first process includes the reference gas supply path R2, the first relay path R3, the second relay path R4, and the first exhaust path R5. That is to say, the first route follows the reference gas inlet port P2, through which the reference gas G2 is introduced, the first passages 31, 41, the first branch valve 75, the sensor chamber 100, the second branch valve 78, and the second passages 32, 42 in this order.

The second route formed by the second process includes the sample gas supply path R1, the first relay path R3, the second relay path R4, and the second exhaust path R6. That is to say, the second route follows the sample gas inlet port P1, through which the sample gas G1 is introduced, the sample gas supply path R1, the first branch valve 75, the sensor chamber 100, the second branch valve 78, and the gas exhaust path (i.e., the second exhaust path R6) in this order.

The third route formed by the third process includes the reference gas supply path R2, the first relay path R3, the second relay path R4, and the second exhaust path R6. That is to say, the third route follows the reference gas inlet port P2, the first passages 31, 41, the first branch valve 75, the sensor chamber 100, the second branch valve 78, and the gas exhaust path (i.e., the second exhaust path R6) in this order.

In addition, the paths for exhausting the sample gas G1 from the sensor chamber 100 along the second route (namely, the second relay path R4 and the second exhaust path R6) also serve as paths for exhausting the reference gas G2 from the sensor chamber 100 along the third route. That is to say, the paths that the sample gas G1 passes through from the sensor chamber 100 to the gas outlet port P3 along the second route agree with the paths that the reference gas G2 passes through from the sensor chamber 100 to the gas outlet port P3 along the third route.

(2.2) Operation

(2.2.1) Detection Operation

Next, it will be described how the gas detection system 1 according to this embodiment performs the operation of detecting detection target molecules in the sample gas G1. To detect the detection target molecules in the sample gas G1, the gas detection system 1 supplies the reference gas G2, in which the detection target molecules have been reduced significantly (i.e., in which there are almost no detection target molecules), to the gas sensor 11 and acquires, as a reference value, the output value of the gas sensor 11. Thereafter, the gas detection system 1 supplies the sample gas G1 to the gas sensor 11 and calculates the difference between the output value of the gas sensor 11 at this time and the reference value, thereby detecting the detection target molecules in the sample gas G1.

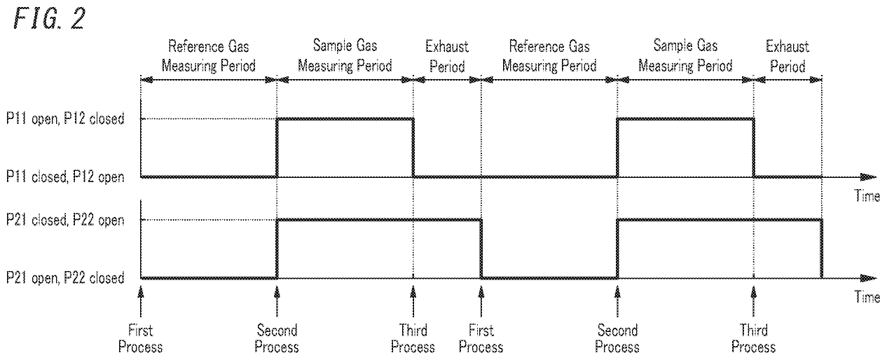

The detection operation to be performed by the gas detection system 1 will now be described with reference to the timing chart shown in FIG. 2.

At the beginning of the detection operation, the control unit 51 starts running the air pump 90. In the first branch valve 75, the input port P12 thereof is normally open. In the second branch valve 78, the first output port P21 thereof is normally open. Thus, after the reference gas G2 introduced through the second air inlet port P2 has entered the sensor chamber 100 via the reference gas supply path R2 and the first relay path R3, the reference gas G2 is exhausted from the sensor chamber 100 to the outside through the gas outlet port P3 via the second relay path R4 and the first exhaust path R5.

The control unit 51 according to this embodiment goes through a reference gas measuring period in which the reference gas G2 is supplied to the sensor chamber 100 to acquire a reference value, a sample gas measuring period in which the sample gas G1 is supplied to the sensor chamber 100 to detect the detection target molecules, and an exhaust period in which the reference gas G2 is supplied to the sensor chamber 100 to exhaust the sample gas G1 in this order. After having gone through the exhaust period, the control unit 51 goes through the reference gas measuring period again.

(Reference Gas Measuring Period)

In the reference gas measuring period, the control unit 51 operates the first branch valve 75 to open the second input port P12 thereof and close the first input port P11 thereof, and also operates the second branch valve 78 to open the first output port P21 thereof and close the second output port P22 thereof (i.e., performs the first process). This allows the gas flow channels to be changed such that after the reference gas G2 has been introduced through the second air inlet port P2 into the sensor chamber 100 via the reference gas supply path R2 and the first relay path R3, the reference gas G2 is exhausted from the sensor chamber 100 via the second relay path R4 and the first exhaust path R5. When the control unit 51 starts running the air pump 90 in this state, the reference gas G2 that has been sucked in through the second air inlet port P2 is introduced into the sensor chamber 100 via the reference gas supply path R2 and the first relay path R3 and then exhausted through the gas outlet port P3 via the second relay path R4 and the first exhaust path R5.

In the reference gas measuring period, the reference gas G2 introduced through the second air inlet port P2 has dust and other particles with a relatively large particle size filtered out by the particle filter 72, and then is passed through the check valve 73 and supplied to the first passage 31 of the first filter 30. While the reference gas G2 is flowing through the first passage 31 of the first filter 30, the VOCs as the target components to be removed in the reference gas G2 permeate into the separation membrane, because the second passage 32 has a lower pressure than the first passage 31. The VOCs permeating into the separation membrane diffuses through the separation membrane toward the second passage 32, leaves the separation membrane, and then enters the second passage 32. Thereafter, the VOCs that have entered the second passage 32 will be exhausted from the second passage 32 to the outside through the gas outlet port P3 by way of the pipe 65c, the check valve 79, the pipe 65d, the air pump 90, and the pipe 67 in this order.

Meanwhile, while the reference gas G2 that has passed through the first passage 31 of the first filter 30 is flowing through the first passage 41 of the second filter 40 via the pipe 62d, the water as the target component to be removed in the reference gas G2 permeates into the separation membrane, because the second passage 42 has a lower pressure than the first passage 41. The water permeating into the separation membrane diffuses through the separation membrane toward the second passage 42, leaves the separation membrane, and then enters the second passage 42. Thereafter, the water that has entered the second passage 42 will be exhausted from the second passage 42 to the outside through the gas outlet port P3 by way of the pipe 65b, the second passage 32 of the first filter 30, the pipe 65c, the check valve 79, the pipe 65d, the air pump 90, and the pipe 67 in this order.

As can be seen, the reference gas G2 introduced through the second air inlet port P2 is passed through the first filter 30 and the second filter 40 to have its VOCs and water reduced and then is supplied to the sensor chamber 100 of the housing 10. Speaking more specifically, the process of letting the reference gas G2 pass through the filter unit 20 and then be supplied to the gas sensor 11 includes letting the reference gas G2 pass through the first filter 30 and the second filter 40 in this order and then supplying the reference gas G2 to the sensor chamber 100 (i.e., to the gas sensor 11). Thus, the reference gas G2 that has had its VOCs and water reduced is supplied to the gas sensor 11, and therefore, the output value of the gas sensor 11 in this state may be determined to be a reference for VOCs as detection target molecules. In addition, the reference gas G2 that has had its water reduced is supplied to the gas sensor 11, thus reducing the chances of the water contained in the reference gas G2 causing deterioration to the gas sensor 11 and/or a variation in measurement results.

In addition, the first filter 30 and the second filter 40 each reduce the target components to be removed in the reference gas G2 by using the separation membrane formed out of a hollow fiber, thus hardly allowing the target components to be removed to be accumulated, compared to a filter that uses a filtering material such as an activated charcoal. This significantly reduces the frequency of occurrence of maintenance required.

In this embodiment, the control unit 51 performs, in the reference gas measuring period, the process of controlling the water content of the reference gas G2 by adjusting the valve opening of the proportional control solenoid valve 76 based on the content of water in the reference gas G2 that has been measured by the water content measuring unit 12.

For this purpose, the control unit 51 may compare, for example, the absolute humidity of the reference gas G2 in the sensor chamber 100 as measured by the water content measuring unit 12 with a predetermined reference humidity. The reference humidity may be 1 g/m³, for example, but may be changed as appropriate according to the condition of use, for example.

When finding the absolute humidity of the reference gas G2 in the sensor chamber 100 higher than the reference humidity, the control unit 51 controls the orifice diameter of the proportional control solenoid valve 76 at a first opening diameter. In this case, the first opening diameter is set at a value smaller than the orifice diameter in a situation where the absolute humidity of the reference gas G2 is equal to or less than the reference humidity. Having the control unit 51 control the orifice diameter of the proportional control solenoid valve 76 at the first opening diameter widens the pressure difference before and after the proportional control solenoid valve 76, thus lowering the pressure in the second passage 42 of the second filter 40. This increases the difference in pressure between the first passage 41 and the second passage 42 and thereby causes an increase in the amount of water that moves from the first passage 41 to the second passage 42 in the second filter 40 by permeating through the separation membrane. As a result, the content of water in the reference gas G2 in the sensor chamber 100 decreases to bring the humidity closer toward the predetermined reference humidity.

On the other hand, when finding the absolute humidity of the reference gas G2 in the sensor chamber 100 equal to or lower than the reference humidity, the control unit 51 controls the orifice diameter of the proportional control solenoid valve 76 at a second opening diameter, which is larger than the first opening diameter. In that case, compared to the situation where the orifice diameter of the proportional control solenoid valve 76 is the first opening diameter, the pressure difference before and after the proportional control solenoid valve 76 decreases, thus raising the pressure in the second passage 42 of the second filter 40. This decreases the difference in pressure between the first passage 41 and the second passage 42 and thereby causes a decrease in the amount of water that moves from the first passage 41 to the second passage 42 in the second filter 40 by permeating through the separation membrane. As a result, the content of water in the reference gas G2 in the sensor chamber 100 increases to bring the humidity closer toward the predetermined reference humidity.

As can be seen, the control unit 51 maintains the absolute humidity of the reference gas G2 in the sensor chamber 100 at the reference humidity by adjusting the difference in pressure between the first passage 41 and second passage 42 of the second filter 40. The detection unit 52 acquires, based on the output value of the gas sensor 11 in this state, the reference concentration value of the detection target molecules. In the reference gas measuring period, the quality of the reference gas G2 is controlled with good stability by adjusting the content of water in the reference gas G2. This reduces a dispersion in the reference concentration value of VOCs as detection target molecules, thus allowing the VOCs in the sample gas G1 to be measured more accurately. [0071] (Sample gas measuring period) Next, it will be described how the gas detection system 1 operates in the sample gas measuring period. When a transition is made from the reference gas measuring period to the sample gas measuring period, the control unit 51 operates the first branch valve 75 to open the first input port P11 thereof and close the second input port P12 thereof, and also operates the second branch valve 78 to close the first output port P21 thereof and open the second output port P22 thereof (i.e., performs the second process). This allows the gas flow channels to be changed such that after the sample gas G1 has been introduced through the first air inlet port P1 into the sensor chamber 100 via the sample gas supply path R1 and the first relay path R3, the sample gas G1 is exhausted from the sensor chamber 100 via the second relay path R4 and the second exhaust path R6. When the control unit 51 starts running the air pump 90 in this state, the sample gas G1 that has been sucked in through the first air inlet port P1 is introduced into the sensor chamber 100 via the sample gas supply path R1 and the first relay path R3 and then exhausted through the gas outlet port P3 via the second relay path R4 and the second exhaust path R6.

In the sample gas measuring period, the detection unit 52 acquires the output value of the gas sensor 11 and performs the process of detecting, based on this output value and the reference value that has been acquired during the reference gas measuring period, the detection target molecules in the sample gas G1 (e.g., VOCs as odor molecules in this embodiment). In this embodiment, the detection unit 52 may detect the presence or absence of the detection target molecules. Alternatively, the detection unit 52 may detect the concentration of the detection target molecules. Still alternatively, the detection unit 52 may determine whether the concentration of the detection target molecules is higher or lower than a predetermined setting. In addition, in the sample gas measuring period, the sample gas G1 is exhausted through the second exhaust path R6 that bypasses the filter unit 20, and therefore, the sample gas G1 that has not passed through the first filter 30 does not pass through the second filter 40. This reduces deterioration of the second filter 40 that would be caused by the VOCs.

(Exhaust Period)

Next, it will be described how the gas detection system 1 operates in the exhaust period. When a transition is made from the sample gas measuring period to the exhaust period, the control unit 51 operates the first branch valve 75 to open the second input port P12 thereof and close the first input port P11 thereof, and also operates the second branch valve 78 to open the second output port P22 thereof and close the first output port P21 thereof (i.e., performs the third process). This allows the gas flow channels to be changed such that after the reference gas G2 has been introduced through the second air inlet port P2 into the sensor chamber 100 via the reference gas supply path R2 and the first relay path R3, the reference gas G2 is exhausted from the sensor chamber 100 via the second relay path R4 and the second exhaust path R6. When the control unit 51 starts running the air pump 90 in this state, the reference gas G2 that has been sucked in through the second air inlet port P2 is introduced into the sensor chamber 100 via the reference gas supply path R2 and the first relay path R3 and then exhausted through the gas outlet port P3 via the second relay path R4 and the second exhaust path R6.

In the exhaust period, the detection unit 52 discards the output value of the gas sensor 11. Optionally, in the exhaust period, the control unit 51 may also perform, as in the reference gas measuring period, the process of controlling the water content of the reference gas G2 by adjusting the valve opening of the proportional control solenoid valve 76 based on the content of water in the reference gas G2 as measured by the water content measuring unit 12.

In the exhaust period, the reference gas G2 is supplied to the sensor chamber 100. Thus, the sample gas G1 that has been introduced into the sensor chamber 100 during the sample gas measuring period and still remains in the sensor chamber 100 is pushed out by the reference gas G2 to be exhausted through the gas outlet port P3. In other words, the gas in the sensor chamber 100 is replaced with the reference gas G2. The reference gas G2 to be introduced into the sensor chamber 100 has had its VOCs and water reduced by passing through the filter unit 20. Thus, providing this exhaust period may reduce the VOCs and water in the sensor chamber 100.

In addition, providing the exhaust period may also reduce the chances of residual components of the sample gas G1, including the VOCs and water at relatively high concentrations, passing through the filter unit 20 during the reference gas measuring period. This reduces deterioration in the filtering performance of the filter unit 20.

In the exhaust period, not only the sample gas G1 remaining in the sensor chamber 100 but also the sample gas G1 remaining in the first relay path R3 and the second relay path R4 may be exhausted as well.

The exhaust period is supposed to last for a predetermined period of time. The predetermined period of time may be 3 seconds, for example. When the predetermined period of time passes since the third process has started to be performed at the beginning of the exhaust period, the control unit 51 starts performing the first process to let the reference gas measuring period begin. The predetermined period of time needs to be at least long enough to exhaust the gases in the sensor chamber 100 and the piping. The predetermined period of time may be determined by, for example, the capacities of the sensor chamber 100 and the piping and the flow velocities of the gases.

The control unit 51 of the gas detection system 1 repeatedly performs the detection operation of detecting VOCs in the sample gas G1 by going through the reference gas measuring period, the sample gas measuring period, and the exhaust period in this order in multiple cycles. In this case, from the second cycle and on, the process of controlling the content of water in the reference gas G2 during the reference gas measuring period may be omitted. In addition, in this embodiment, the lengths of the reference gas measuring period and the sample gas measuring period may be set at, for example, 10 seconds. However, the lengths of the reference gas measuring period and the sample gas measuring period may be changed as appropriate. For example, the length of the reference gas measuring period may be set to be different from the length of the sample gas measuring period.

As described above, the gas detection mechanism 60 according to this embodiment includes, separately from the sample gas supply path R1 for supplying the sample gas G1 to the sensor chamber 100, the reference gas supply path R2 for supplying the reference gas G2 to the sensor chamber 100 after letting the reference gas G2 pass through the filter unit 20. The reference gas G2 introduced through the second air inlet port P2 during the reference gas measuring period is supplied to the sensor chamber 100 after having had the detection target molecules (e.g., VOCs in this embodiment) and water reduced by the filter unit 20. This allows the air in the environment to be used as the reference gas G2.

In addition, the gas detection mechanism 60 also includes, separately from the first exhaust path R5 for exhausting the reference gas G2 from the sensor chamber 100, the second exhaust path R6 for exhausting the sample gas G1 from the sensor chamber 100 without letting the sample gas G1 pass through the filter unit 20. This allows the sample gas G1 including the detection target molecules (e.g., VOCs in this embodiment) and water to be exhausted to the outside without passing through the purge line (i.e., the second passages 32, 42) of the filter unit 20, thus reducing deterioration in the filtering performance of the filter unit 20. For example, even if a gas containing water, such as a gas taken from a food or a breath, is used as the sample gas G1, the sample gas G1 is also exhausted to the outside through the second exhaust path R6, thus reducing deterioration in the filtering performance of the filter unit 20 as well.

Furthermore, the reference gas G2 introduced during the exhaust period is exhausted through the second exhaust path R6 while bypassing (i.e., without passing through) the filter unit 20. This reduces deterioration in the filtering performance of the filter unit 20.

Furthermore, the gas detection mechanism 60 includes the proportional control solenoid valve 76 downstream of the sensor chamber 100. The flow rates of the gases (namely, the sample gas G1 and the reference gas G2) passing through sensor chamber 100 may be reduced by decreasing the orifice diameter of the proportional control solenoid valve 76. This reduces not only deterioration in the filtering performance of the filter unit 20 but also deterioration of the gas sensor 11 as well.

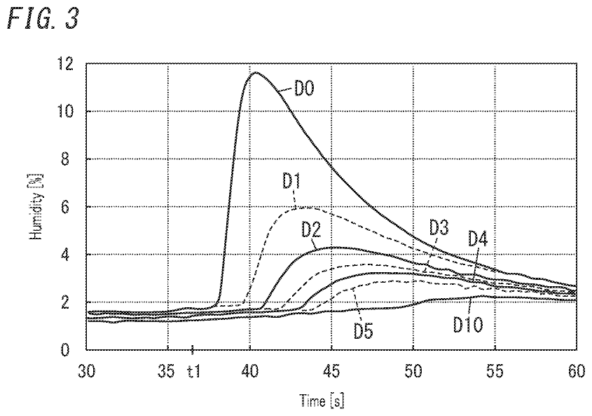

FIG. 3 shows how the humidity changed with time in the pipe 65a between the second branch valve 78 and the second filter 40. In FIG. 3, the curves designated by the reference signs D0, D1, D2, D3, D4, D5, and D10 show how the humidity changed with time in seven different situations where the lengths of the exhaust period were 0 seconds, 1 second, 2 seconds, 3 seconds, 4 seconds, 5 seconds, and 10 seconds, respectively.

The period prior to a time t1 (of about 36.5 seconds) is the sample gas measuring period. The curve D0 shows the results obtained when the reference gas measuring period was allowed to begin at the time t1 with no exhaust period provided. The other curves D1, D2, D3, D4, D5, and D10 show the results obtained when the exhaust period was allowed to begin at the time t1. In this case, the reference gas measuring period was allowed to begin when the exhaust period ended.

At the beginning of the reference gas measuring period, the first output port P21 of the second branch valve 78 is opened, when the gases start to be supplied to the pipe 65a. The gases supplied to the pipe 65a may include the sample gas G1 remaining in the sensor chamber 100, for example. The sample gas G1 has not passed through the filter unit 20, and therefore, has a higher humidity than the reference gas G2. Thus, the humidity in the pipe 65a may start to rise from the time t1 on as shown in FIG. 3.

In this case, providing the exhaust period that begins at the time t1 reduces an increase in humidity, compared to a situation where no exhaust period is provided (as indicated by the curve D0). That is to say, in the former case, the sample gas G1 remaining in the sensor chamber 100, for example, is exhausted during the exhaust period, thus reducing the chances of the sample gas G1 being introduced into the pipe 65a and thereby reducing an increase in humidity. Consequently, this reduces deterioration of the filter unit 20 that would be caused if the sample gas G1 passed through the filter unit 20.

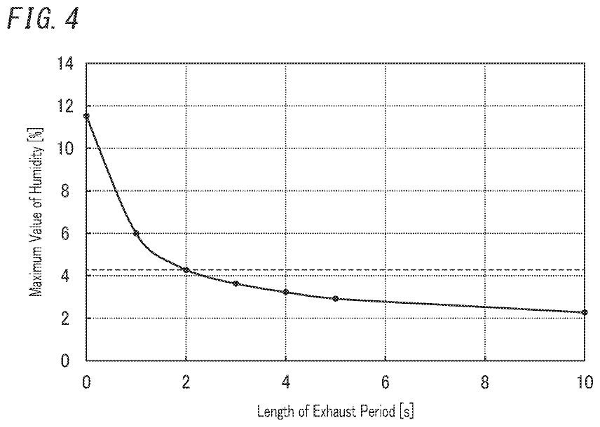

The maximum value of the humidity in the pipe 65a was obtained based on the data shown in FIG. 3. The results are shown in FIG. 4. When no exhaust period was provided (i.e., when the length of the exhaust period was 0 seconds), the maximum value of the humidity was about 12%. The longer the exhaust period was, the smaller the maximum value of the humidity was. In FIG. 4, a value which is 1/e (where e is a Napier's constant) as large as the maximum humidity when no exhaust period is provided is indicated by the dotted line. As shown in FIG. 4, if the length of the exhaust period is set to be equal to or longer than about 2 seconds, then the maximum humidity becomes at most 1/e as large as the maximum humidity when no exhaust period is provided. That is why the length of the exhaust period is preferably equal to or longer than 2 seconds, for example.

(2.2.2) Sleep Mode

Next, a sleep mode in which the gas detection system 1 according to this embodiment suspends performing the operation of detecting the sample gas G1 will be described.

In the sleep mode, the control unit 51 operates the first branch valve 75 to open the second input port P12 thereof and close the first input port P11 thereof and also operates the second branch valve 78 to open the first output port P21 thereof and close the second output port P22 thereof.

In that case, the first branch valve 75 prevents the outside air from flowing in through the first air inlet port P1. The check valve 73 and the check valve 74 prevent the outside air from flowing in through the second air inlet port P2. The check valve 79, the second branch valve 78, and the check valve 77 prevent the outside air from flowing in through the gas outlet port P3. According to this embodiment, a plurality of flow channels (namely, the sample gas supply path R1, the reference gas supply path R2, the first exhaust path R5, and the second exhaust path R6) may connect the filter unit 20 to the outside. These flow channels are provided, as cutoff elements, the first branch valve 75, the second branch valve 78, and the check valves 73, 74, 77 and 79. In the sleep mode, the plurality of flow channels are cut off from the sensor chamber 100 by these cutoff elements. In other words, a cutoff section CB1 including the plurality of cutoff elements cuts off the filter unit 20 from the outside in the sleep mode (e.g., in a power OFF state). This reduces the exposure of the filter unit 20 to the outside air, thus reducing deterioration of the filter unit 20. Note that the cutoff section CB1 includes solenoid valves (namely, the first branch valve 75 and the second branch valve 78) to be opened or closed with electromagnetic force, thus allowing the solenoid valves to be opened or closed in response to an electrical signal supplied from the control unit 51. In addition, the cutoff section CB1 includes the check valve 79, thus achieving the advantage of eliminating the need to use a power supply or signal lines unlike a situation where the solenoid valves are used.

Optionally, when a transition is made to the sleep mode, the control unit 51 may operate the first branch valve 75 to open the second input port P12 thereof and close the first input port P11 thereof and activate the air pump 90 to reduce the pressure in the circuit, including the filter unit 20, to a pressure lower than the external pressure (e.g., to a vacuum). The control unit 51 stops running the air pump 90 after having activated the air pump 90 for a predetermined period of time, thereby holding the pressure in the circuit section, including the filter unit 20 that has been cut off by the cutoff section CB1, at a negative pressure. In the sleep mode, reducing the pressure in the circuit C1 leading from the first branch valve 75 to the check valve 79 via the housing 10 makes the check valve 79 openable less easily, thus reducing the chances of the filter unit 20 being exposed to the outside air.

(2.2.3) Idle Operation

The gas detection system 1 according to this embodiment may go through an idle period in which the gas detection system 1 performs an idle operation including letting the reference gas G2 flow through the filter unit 20 in the sleep mode.

In the idle period, the reference gas G2 introduced through the second air inlet port P2 is supplied to the sensor chamber 100 via the reference gas supply path R2 and then exhausted to the outside via the first exhaust path R5. The hollow fiber separation membrane included in the second filter 40 has filter performance that significantly varies according to its degree of dryness. Letting the separation membrane contain a lot of moisture owing to a long duration of the sleep mode could cause a decline in the filter performance. Thus, letting the reference gas G2 flow through the filter unit 20 in the idle period enables drying the hollow fiber separation membrane included in the second filter 40, which would improve the filter performance of the second filter 40.

In this embodiment, when finding the water content detected in the sleep mode by the detection unit 52 based on the result of measurement by the water content measuring unit 12 higher than a predetermined first threshold value, the control unit 51 starts performing the idle operation by activating the air pump 90. Thereafter, when finding the water content detected by the detection unit 52 equal to or less than a predetermined second threshold value, the control unit 51 stops running the air pump 90 to maintain the circuit section, including the filter unit 20 cut off by the cutoff section CB1, in a negative pressure state.

In this manner, the gas detection system 1 performs the idle operation when finding the water content detected in the sleep mode by the detection unit 52 higher than the first threshold value, thus maintaining the second filter 40 in the dry condition. This allows the gas detection system 1 to resume the detection operation with the second filter 40 kept dry.

In this case, the control unit 51 preferably controls the orifice diameter of the proportional control solenoid valve 76 to make the flow rate of the reference gas G2 when the idle operation is performed higher than the flow rate of the reference gas G2 in the reference gas measuring period when the detection operation is performed. Letting the reference gas G2 flow at a high flow rate in the idle period in this manner allows the filter unit 20 to be dried in a short time to end the idle operation quickly.

In this embodiment, the control unit 51 determines, based on the result of measurement by the water content measuring unit 12, the timings to start and end the idle operation. However, this is only an example and should not be construed as limiting. Alternatively, the control unit 51 may determine the timings to start and end the idle operation based on the output value of the gas sensor 11 that varies according to the content of water in the gas. That is to say, supposing the content of VOCs in the gas is constant, the larger the content of water in the gas is, the lower the VOC concentration to be detected based on the output value of the gas sensor 11 is.

If the control unit 51 determines the timings to start and end the idle operation based on the output value of the gas sensor 11, which varies according to the content of water in the gas, then the gas detection system 1 does not have to include the water content measuring unit 12. The control unit 51 estimates, in the reference gas measuring period, the content of water in the gas based on the output value of the gas sensor 11 and controls the operation of the air pump 90 based on the water content thus estimated. This stabilizes the quality of the reference gas G2 and thereby reduces a dispersion in the detection results of the sample gas G1. Also, the characteristics of the gas sensor 11 may either vary or deteriorate with time. Nevertheless, supplying a reference gas G2, having a reduced VOC concentration and reduced water content, to the gas sensor 11 in the idle period allows the gas sensor 11 to recover its initial characteristics.

### (3) Variations

Note that the embodiment described above is only an exemplary one of various embodiments of the present disclosure and should not be construed as limiting. Rather, the exemplary embodiment may be readily modified in various manners depending on a design choice or any other factor without departing from the scope of the present disclosure. Also, the functions of the control system 50 may also be implemented as a method for controlling the gas detection mechanism 60, a computer program, or a non-transitory storage medium on which the program is stored.

Next, variations of the exemplary embodiment will be enumerated one after another. Note that the variations to be described below may be adopted in combination as appropriate.

The gas detection system 1 according to the present disclosure includes a computer system as the control system 50, for example. The computer system may include a processor and a memory as principal hardware components thereof. The functions of the gas detection system 1 according to the present disclosure may be performed by making the processor execute a program stored in the memory of the computer system. The program may be stored in advance in the memory of the computer system. Alternatively, the program may also be downloaded through a telecommunications line or be distributed after having been recorded in some non-transitory storage medium such as a memory card, an optical disc, or a hard disk drive, any of which is readable for the computer system. The processor of the computer system may be made up of a single or a plurality of electronic circuits including a semiconductor integrated circuit (IC) or a large-scale integrated circuit (LSI). As used herein, the “integrated circuit” such as an IC or an LSI is called by a different name depending on the degree of integration thereof. Examples of the integrated circuits include a system LSI, a very-large-scale integrated circuit (VLSI), and an ultra-large-scale integrated circuit (ULSI). Optionally, a field-programmable gate array (FPGA) to be programmed after an LSI has been fabricated or a reconfigurable logic device allowing the connections or circuit sections inside of an LSI to be reconfigured may also be adopted as the processor. Those electronic circuits may be either integrated together on a single chip or distributed on multiple chips, whichever is appropriate. Those multiple chips may be aggregated together in a single device or distributed in multiple devices without limitation. As used herein, the “computer system” includes a microcontroller including one or more processors and one or more memories. Thus, the microcontroller may also be implemented as a single or a plurality of electronic circuits including a semiconductor integrated circuit or a large-scale integrated circuit.

Also, in the embodiment described above, the plurality of functions of the gas detection system 1 are integrated together in a single housing. However, this is not an essential configuration for the gas detection system 1. Alternatively, those constituent elements of the gas detection system 1 may be distributed in multiple different housings. For example, a housing that integrates together the functions of the control system 50 may be provided separately from a housing that integrates together the functions of the gas detection mechanism 60. Still alternatively, at least some functions of the gas detection system 1 (e.g., some functions of the control system 50) may be implemented as, for example, a cloud computing system as well.

In the foregoing description of embodiments, if one of two values of measurement data, for example, being compared with each other is “greater than” the other, the phrase “greater than” may also be a synonym of the phrase “equal to or greater than.” That is to say, it is arbitrarily changeable, depending on selection of a reference value or any preset value, whether the phrase “greater than” covers the situation where the two values are equal to each other. Therefore, from a technical point of view, there is no difference between the phrase “greater than” and the phrase “equal to or greater than.” Similarly, the phrase “equal to or less than” may be a synonym of the phrase “less than” as well in the embodiment described above.

In the exemplary embodiment described above, the proportional control solenoid valve 76 is disposed upstream of (i.e., closer to the housing 10 than) the second branch valve 78. This proportional control solenoid valve 76 may be replaced with two proportional control solenoid valves respectively arranged downstream of the first output port P21 and second output port P22 of the second branch valve 78. That is to say, a first proportional control solenoid valve may be disposed between the first output port P21 of the second branch valve 78 and the filter unit 20 to control the flow rate of the gas flowing through the first exhaust path R5. In addition, a second proportional control solenoid valve may be disposed between the second output port P22 of the second branch valve 78 and a confluent portion of the pipes 66 and 65d to control the flow rate of the gas flowing through the second exhaust path R6.

In the exemplary embodiment described above, the check valve 73 is disposed between the second air inlet port P2 and the filter unit 20. Alternatively, a two-way solenoid valve may be connected there instead of the check valve 73.

In the exemplary embodiment described above, the check valve 77 is disposed between the proportional control solenoid valve 76 and the second branch valve 78. Alternatively, a two-way solenoid valve may be disposed instead of the check valve 77. Replacing the check valve 77 with the two-way solenoid valve may prevent the two-way solenoid valve from opening unintentionally, thus increasing the degree of airtightness in the circuit section including the filter unit 20 in the sleep mode. Optionally, in the exemplary embodiment described above, the check valves 74 and 79 may each be replaced with a two-way solenoid valve.

Also, the check valve 77 connected between the proportional control solenoid valve 76 and the second branch valve 78 in the exemplary embodiment described above may be removed.

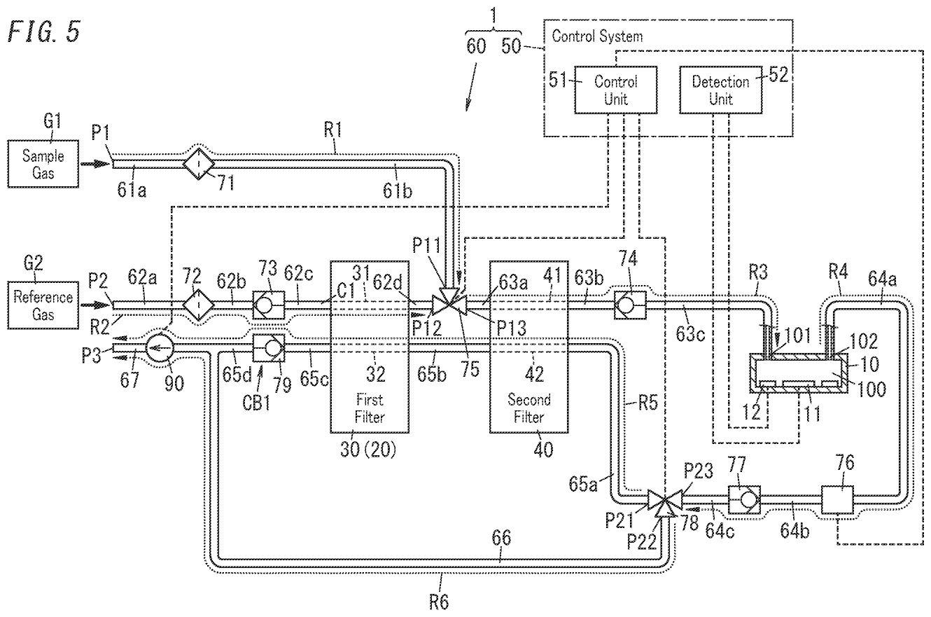

Furthermore, in the exemplary embodiment described above, the sample gas supply path R1 runs through neither the first filter 30 nor the second filter 40. Alternatively, as in a first variation shown in FIG. 5, the sample gas supply path R1 may also be configured to run through only the second filter 40 that filters out water, out of the first filter 30 and the second filter 40. In that case, the filter unit 20 refers to a configuration consisting of only the first filter 30, not including the second filter 40.

In the gas detection mechanism 60 shown in FIG. 5, the second input port P12 of the first branch valve 75 is connected to the first passage 31 of the first filter 30 via the pipe 62d and the output port P13 of the first branch valve 75 is connected to one end of the first passage 41 of the second filter 40 via a pipe 63a. The other end of the first passage 41 of the second filter 40 is connected to the input port of the check valve 74 via a pipe 63b. The output port of the check valve 74 is connected to the first port 101 of the housing 10 via a pipe 63c.

In this gas detection mechanism 60, letting the sample gas G1 pass through the second filter 40 reduces the content of water in the sample gas G1, thus reducing the chances of the output value of the gas sensor 11 being affected by the water in the sample gas G1 and thereby reducing a measurement error to be caused by the water. In addition, in the exhaust period, the reference gas G2 does not pass through the first filter 30 (filter unit 20), thus reducing deterioration of the first filter 30.

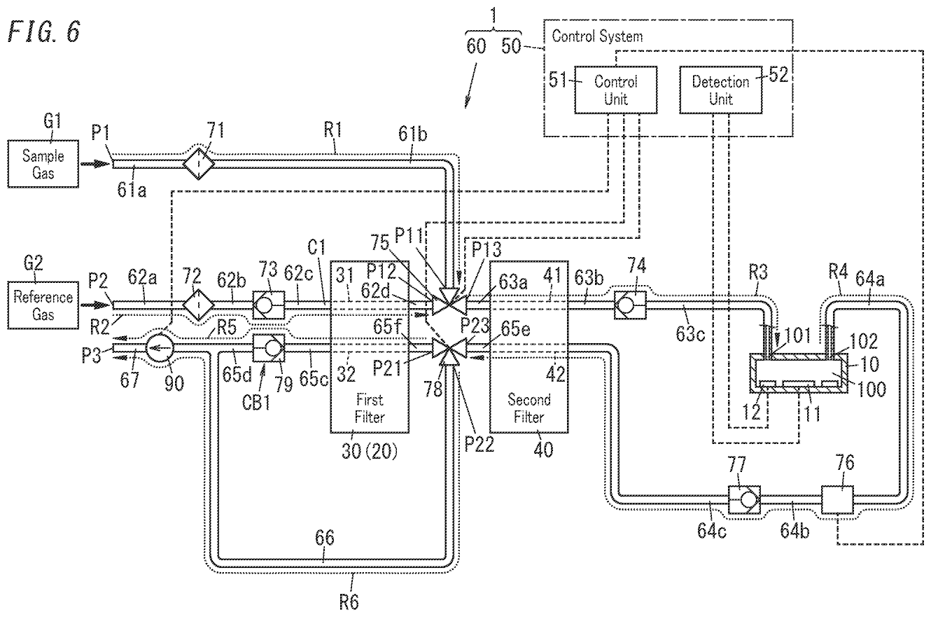

Furthermore, in the gas detection mechanism 60 shown in FIG. 5, the second exhaust path R6 for exhausting the sample gas G1 runs through neither the first filter 30 nor the second filter 40. Alternatively, as in a second variation shown in FIG. 6, the second exhaust path R6 may also be configured to run through only the second filter 40 that filters out water, out of the first filter 30 and the second filter 40. In that case, the filter unit 20 also refers to a configuration consisting of only the first filter 30, not including the second filter 40.

In the gas detection mechanism 60 shown in FIG. 6, the second passage 42 of the second filter 40 is connected to the input port P23 of the second branch valve 78 via a pipe 65e and the first output port P21 of the second branch valve 78 is connected to the second passage 32 of the first filter 30 via a pipe 65f. The output port of the check valve 77 is connected to the second passage 42 of the second filter 40 via the pipe 64c.

According to this configuration, the sample gas G1 introduced through the first air inlet port P1 is supplied to the sensor chamber 100 through the first passage 41 of the second filter 40 and then exhausted from the sensor chamber 100 to the outside through the gas outlet port P3 via the second passage 42 of the second filter 40. This gas detection mechanism 60 may reduce the content of water in the sample gas G1 thus reducing the chances of the output value of the gas sensor 11 being affected by the water in the sample gas G1 and thereby reducing a measurement error to be caused by the water. In other words, the sample gas G1 has its water content reduced by the second filter 40 and the reference gas G2 has its VOC concentration and water content reduced by the first filter 30 and the second filter 40, respectively. Thus, the output value of the gas sensor 11 in a state where the sample gas G1 is supplied to the sensor chamber 100 will correspond to the VOCs in the sample gas G1. This allows the gas detection system 1 to detect the VOCs more accurately.

In the exemplary embodiment described above, the proportional control solenoid valve 76 is disposed as a variable orifice upstream of (i.e., closer to the housing 10 than) the second branch valve 78. Alternatively, the proportional control solenoid valve 76 may be replaced with a speed control valve for controlling the flow rate at a predetermined value. Providing the speed control valve instead of the proportional control solenoid valve 76 enables changing the flow rate downstream of the speed control valve without changing the flow rate of the gas flowing through the sensor chamber 100. This stabilizes the output value of the gas sensor 11, thus enabling making measurement with good stability.

In the exemplary embodiment described above, the control unit 51 adjusts the content of water in the reference gas G2 passing through the filter unit 20 by changing the flow rate of the reference gas G2 based on the result of measurement of the content of water in the reference gas G2. Alternatively, a measuring condition other than the flow rate of the reference gas G2 may be changed. For example, the control unit 51 may also adjust the water content (relative humidity) of the reference gas G2 passing through the filter unit 20 by changing, using a heater, for example, the temperature of the reference gas G2 passing through the filter unit 20, based on the result of measurement of the content of water in the reference gas G2.

In the exemplary embodiment described above, the control unit 51 may control, based on the output value of the gas sensor 11, the flow rate of the reference gas G2 flowing through the filter unit 20 in the reference gas measuring period, thus enabling supplying, to the gas sensor 11, a reference gas G2 including almost no detection target molecules. This allows the gas detection system 1 to acquire a reference value with the effect of the detection target molecules reduced in the reference gas measuring period, thus improving the measurement accuracy of the detection target molecules in the sample gas measuring period.

In the exemplary embodiment described above, a gas adsorbent, including a set of plurality of nanowires, may be provided in the sensor chamber 100. The gas may be condensed by having the gas introduced into the sensor chamber 100 adsorbed into the gas adsorbent and the gas desorbed from the gas adsorbent may be supplied to the gas sensor 11. In this manner, the gas detection sensitivity may be increased.

In the exemplary embodiment described above, the reference gas measuring period in which the reference gas G2 is introduced into the sensor chamber 100 and the sample gas measuring period in which the sample gas G1 is introduced into the sensor chamber 100 are temporally separated from each other. Alternatively, a flow channel leading from the sample gas inlet port P1 to the sensor chamber 100 and a flow channel leading from the reference gas inlet port P2 to the sensor chamber 100 may be spatially separated from each other. In that case, each flow channel may be provided with a two-way valve.

In addition, a flow channel leading from the sensor chamber 100 to the gas outlet port P3 via the filter unit 20 and a flow channel leading from the sensor chamber 100 to the gas outlet port P3 while bypassing the filter unit 20 may be spatially separated from each other. In that case, each flow channel may be provided with a two-way valve.

In the exemplary embodiment described above, the first filter 30 for reducing VOCs and the second filter 40 for reducing water are connected together in series. Alternatively, if the separation membrane of the second filter 40 is made of a material that hardly deteriorates even when exposed to VOCs, the first filter 30 and the second filter 40 may also be connected in parallel.

In the exemplary embodiment described above, the filter unit 20 includes the first filter 30 for reducing VOCs and the second filter 40 for reducing water. Alternatively, the filter unit 20 may also be configured as a single filter including a hollow fiber separation membrane that reduces both VOCs and water. For example, VOCs may also be reduced by the second filter 40 for reducing water. Thus, the filter unit 20 may consist of only the second filter 40, out of the first filter 30 and the second filter 40.

The control unit 51 may determine, based on the output value of the water content measuring unit 12, the length of the exhaust period in which the reference gas G2 follows the third route. For example, after letting the exhaust period begin by starting performing the third process, the control unit 51 may allow the exhaust period to continue until the content of water in the reference gas G2 as measured by the water content measuring unit 12 becomes equal to or less than a predetermined threshold value.

In the exemplary embodiment described above, each of the first branch valve 75 and the second branch valve 78 is a three-way branch valve, more specifically, a three-way solenoid valve. Alternatively, mechanical three-way branch valves may also be used as the first branch valve 75 and the second branch valve 78.

In the exemplary embodiment described above, the detection unit 52 discards the output value of the gas sensor 11 in the exhaust period. Alternatively, the detection unit 52 may process the output value of the gas sensor 11 as an effective output value in the exhaust period. That is to say, the detection unit 52 may acquire, as reference values, the output values of the gas sensor 11 in both the exhaust period and the reference gas measuring period. Thereafter, the detection unit 52 may detect the detection target molecules in the sample gas G1 based on the output value of the gas sensor 11 in the sample gas measuring period in which the sample gas G1 is supplied to the sensor chamber 100 and the reference value described above.

The gas detection mechanism 60 does not have to include the air pump 90. Also, the position of the air pump 90 may be changed as appropriate.

The gas detection mechanism 60 does not have to include the particle filters 71, 72.

(Recapitulation)

The exemplary embodiment and its variations described above are specific implementations of the following aspects of the present disclosure.

A gas detection method according to a first aspect is designed to use a gas detection mechanism (60). The gas detection mechanism (60) includes a filter unit (20) and a sensor chamber (100). The filter unit (20) has a first passage (31, 41) and a second passage (32, 42) as a gas flow channel. The filter unit (20) selectively transfers a component to be removed in a gas from the first passage (31, 41) to the second passage (32, 42). The sensor chamber (100) houses a gas sensor (11). The gas detection method includes a first process, a second process, and a third process. The first process includes changing routes to be followed, from inlet to outlet, by a reference gas (G2) into a first route. The reference gas (G2) provides a reference concentration for detection target molecules in a sample gas (G1). The first route follows the first passage (31, 41), the sensor chamber (100), and the second passage (32, 42) in this order. The second process includes changing routes to be followed, from inlet to outlet, by the sample gas (G1) into a second route. The second route follows a sample gas supply path (R1) that bypasses the filter unit (20) and the sensor chamber (100) in this order and allows the sample gas to be exhausted without letting the sample gas pass through the filter unit (20) while flowing from the sensor chamber (100). The third process is performed after the second process and before the first process. The third process includes changing routes to be followed, from inlet to outlet, by the reference gas (G2) into a third route. The third route follows the first passage (31, 41) and the sensor chamber (100) in this order and allows the reference gas to be exhausted without letting the reference gas pass through the filter unit (20) while flowing from the sensor chamber (100).

According to this aspect, performing the third process after the second process allows the sample gas (G1) remaining in the sensor chamber (100) to be replaced with the reference gas (G2). This reduces deterioration of the filter unit (20) due to the passage of the sample gas (G1), remaining in the sensor chamber (100), through the second passage (32, 42) while the first process is being performed.

In a gas detection method according to a second aspect, which may be implemented in conjunction with the first aspect, an exhaust path (second relay path R4 and second exhaust path R6) through which the sample gas (G1) is exhausted from the sensor chamber (100) along the second route serves as an exhaust path through which the reference gas (G2) is exhausted from the sensor chamber (100) along the third route.

This aspect allows the sample gas (G1) remaining in the exhaust path to be pushed out and exhausted by the reference gas (G2) during the third process.

In a gas detection method according to a third aspect, which may be implemented in conjunction with the second aspect, the first route follows a reference gas inlet port (P2), the first passage (31, 41), a first branch valve (75), the sensor chamber (100), a second branch valve (78), and the second passage (32, 42) in this order. The reference gas (G2) is introduced through the reference gas inlet port (P2). The second route follows a sample gas inlet port (P1), the sample gas supply path (R1), the first branch valve (75), the sensor chamber (100), the second branch valve (78), and a gas exhaust path (second exhaust path R6) in this order. The sample gas (G1) is introduced through the sample gas inlet port (P1). The third route follows the reference gas inlet port (P2), the first passage (31, 41), the first branch valve (75), the sensor chamber (100), the second branch valve (78), and the gas exhaust path (second exhaust path R6) in this order.

This aspect allows the first, second, and third process to be performed by operating the first branch valve (75) and the second branch valve (78).

In a gas detection method according to a fourth aspect, which may be implemented in conjunction with any one of the first to third aspects, the filter unit (20) includes a separation membrane. The separation membrane includes a hollow fiber. The separation membrane separates the first passage (31, 41) and the second passage (32, 42) from each other.

According to this aspect, the target component to be removed is hardly accumulated in the filter unit (20) unlike a filter that uses a filtering material such as an activated charcoal, thus significantly reducing the frequency of occurrence of maintenance required.

Note that the features according to the second to fourth aspects are not essential features for the gas detection method but may be omitted as appropriate.

A program according to a fifth aspect is designed to cause one or more processors to perform the gas detection method according to any one of the first to fourth aspects.

This aspect reduces deterioration of the filter unit (20).

A control system (50) according to a sixth aspect controls a gas detection mechanism (60). The gas detection mechanism (60) includes a filter unit (20) and a sensor chamber (100). The filter unit (20) has a first passage (31, 41) and a second passage (32, 42) as a gas flow channel. The filter unit (20) selectively transfers a component to be removed in a gas from the first passage (31, 41) to the second passage (32, 42). The sensor chamber (100) houses a gas sensor (11). The control system (50) includes a control unit (51) that performs a first process, a second process, and a third process. The first process includes changing routes to be followed, from inlet to outlet, by a reference gas (G2) into a first route. The reference gas (G2) provides a reference concentration for detection target molecules in a sample gas (G1). The first route follows the first passage (31, 41), the sensor chamber (100), and the second passage (32, 42) in this order. The second process includes changing routes to be followed, from inlet to outlet, by the sample gas (G1) into a second route. The second route follows a sample gas supply path (R1) that bypasses the filter unit (20) and the sensor chamber (100) in this order and allows the sample gas to be exhausted without letting the sample gas pass through the filter unit (20) while flowing from the sensor chamber (100). The third process is performed after the second process and before the first process. The third process includes changing routes to be followed, from inlet to outlet, by the reference gas (G2) into a third route. The third route follows the first passage (31, 41) and the sensor chamber (100) in this order and allows the reference gas to be exhausted without letting the reference gas pass through the filter unit (20) while flowing from the sensor chamber (100).

This configuration may reduce deterioration of the filter unit (20).

A gas detection system (1) according to a seventh aspect includes the control system (50) according to the sixth aspect and the gas detection mechanism (60).

This configuration may reduce deterioration of the filter unit (20).

Note that these are not the only aspects of the present disclosure but various configurations of the gas detection system (1) according to the exemplary embodiment (including its variations) may also be implemented as a gas detection method and a program.

### REFERENCE SIGNS LIST

1 Gas Detection System
11 Gas Sensor
20 Filter Unit
31, 41 First Passage
32,42 Second Passage
50 Control System
51 Control Unit
60 Gas Detection Mechanism
75 First Branch Valve
78 Second Branch Valve
100 Sensor Chamber
G1 Sample Gas
G2 Reference Gas
P1 Sample Gas Inlet Port
P2 Reference Gas Inlet Port
R1 Sample Gas Supply Path
R4 Second Relay Path (Exhaust Path)
R6 Second Exhaust Path (Exhaust Path, Gas Exhaust Path)

1. A gas detection method designed to use a gas detection mechanism, the gas detection mechanism including: a filter unit; and a sensor chamber housing a gas sensor, the filter unit having a first passage and a second passage as a gas flow channel and configured to selectively transfer a component to be removed in a gas from the first passage to the second passage, the gas detection method comprising:
a first process including changing routes to be followed, from inlet to outlet, by a reference gas, providing a reference concentration for detection target molecules in a sample gas, into a first route, the first route following the first passage, the sensor chamber, and the second passage in this order;
a second process including changing routes to be followed, from inlet to outlet, by the sample gas into a second route, the second route following a sample gas supply path and the sensor chamber in this order, the second route allowing the sample gas to be exhausted without letting the sample gas pass through the filter unit while flowing from the sensor chamber, the sample gas supply path bypassing the filter unit; and
a third process including changing, after the second process and before the first process, routes to be followed, from inlet to outlet, by the reference gas into a third route, the third route following the first passage and the sensor chamber in this order, the third route allowing the reference gas to be exhausted without letting the reference gas pass through the filter unit while flowing from the sensor chamber.

2. The gas detection method of claim 1, wherein
an exhaust path through which the sample gas is exhausted from the sensor chamber along the second route serves as an exhaust path through which the reference gas is exhausted from the sensor chamber along the third route.

3. The gas detection method of claim 2, wherein
the first route follows a reference gas inlet port, the first passage, a first branch valve, the sensor chamber, a second branch valve, and the second passage in this order, the reference gas being introduced through the reference gas inlet port,
the second route follows a sample gas inlet port, the sample gas supply path, the first branch valve, the sensor chamber, the second branch valve, and a gas exhaust path in this order, the sample gas being introduced through the sample gas inlet port, and
the third route follows the reference gas inlet port, the first passage, the first branch valve, the sensor chamber, the second branch valve, and the gas exhaust path in this order.

4. The gas detection method of claim 1, wherein
the filter unit includes a separation membrane including a hollow fiber, and
the separation membrane separates the first passage and the second passage from each other.

5. A non-transitory computer-readable storage medium having stored thereon a computer program designed to cause one or more processors of the computer to perform the gas detection method of claim 1.

6. A control system configured to control a gas detection mechanism, the gas detection mechanism including: a filter unit; and a sensor chamber housing a gas sensor, the filter unit having a first passage and a second passage as a gas flow channel and configured to selectively transfer a component to be removed in a gas from the first passage to the second passage, the control system including a control unit configured to perform:
a first process including changing routes to be followed, from inlet to outlet, by a reference gas, providing a reference concentration for detection target molecules in a sample gas, into a first route, the first route following the first passage, the sensor chamber, and the second passage in this order;
a second process including changing routes to be followed, from inlet to outlet, by the sample gas into a second route, the second route following a sample gas supply path and the sensor chamber in this order, the second route allowing the sample gas to be exhausted without letting the sample gas pass through the filter unit while flowing from the sensor chamber, the sample gas supply path bypassing the filter unit; and
a third process including changing, after the second process and before the first process, routes to be followed, from inlet to outlet, by the reference gas into a third route, the third route following the first passage and the sensor chamber in this order, the third route allowing the reference gas to be exhausted without letting the reference gas pass through the filter unit while flowing from the sensor chamber.

7. A gas detection system comprising:
the control system of claim 6; and
the gas detection mechanism.
